(19) United States
(12) Patent Application Publication
(10) Pub. No.: US 2010/0291115 A1
(43) Pub. Date: Nov. 18, 2010

(54) PHARMACEUTICAL COMPOSITION FOR THE SUBLINGUAL ADMINISTRATION OF VACCINES, METHOD FOR THE PREPARATION OF THE SAME AND USES THEREOF

(76) Inventors: Bruno Silvestrini, Roma (IT); Michele Bonanomi, Roma (IT)

Correspondence Address: Pearne & Gordon LLP, 1801 East 9th Street, Suite 1200, Cleveland, OH, 44114-3108, US

(21) Appl. No.: 12/303,726

(22) PCT Filed: Jun. 6, 2007
(86) PCT No.: PCT/IB07/01521

§ 371 Date: Dec. 6, 2008

(30) Foreign Application Priority Data
Jun. 9, 2006 (IT) MI2006A001117

(51) Int. Cl. A61K 9/00; A61K 39/00; A61P 37/04; A61P 31/12; A61P 31/04; A61P 31/16

(52) U.S. Cl. 424/184.1

(57) ABSTRACT

The subject of the present invention is a pharmaceutical composition for the sublingual administration of vaccines, a method for its preparation and the use thereof for modulating the absorption rate and substantially improving its effectiveness and safety. Said sublingual composition is applicable both to immunizing vaccines, which are employed against infections, and to desensitising vaccines, which have allergies as therapeutic indication.

The subject of the present invention is a pharmaceutical composition for the sublingual administration of vaccines, a method for the preparation of the same and its use for modulating the absorption rate and substantially improving the effectiveness and the safety. Said composition is applicable for sublingually administering both immunizing vaccines, which are employed against infections, and desensitising vaccines, which have allergies as a therapeutic indication.

It is known that living organisms defend themselves from attacks of pathogenic agents not making use of a specialized apparatus, but by mobilizing their general processes.

The two more typical biological expressions of this defensive strategy are the inflammation and the so-called acquired immunity. The former derives from a diffuse mobilization of the organism defenses, which is manifested in the variety of its symptoms (reddening, pain; fever and swelling, to which the temporary paralysis of the affected part is added), each of which expresses the sharpening of a physiological process, starting, for example, from those involved in the tissues irritation, the body temperature and the sensitive-sensorial perception of stimuli/signals required for their regulation. The only drugs currently available in order to combat the inflammation are the anti-inflammatory agents, consisting of hundreds of steroidal and non-steroidal compounds.

Unlike the inflammation, the acquired immunity only concerns the immune system of the organism which, during the phylogeny, is become specialized in the task of differentiating and keeping separate the “oneself” from the “non oneself” (where by the aforesaid terms it is to be intended herein what concerns the functioning and the composition of the organism and what is unrelated thereto, respectively). The immune system is always functioning, but it becomes hyperactive in emergency situations, such as those typically represented by the infections. This hyperactivation usually requires a latent period which is about 2-3 weeks, but when it is completed offers a particularly marked protection, allowing the organism to withstand loadings of the particular microbe or toxin against which it has mobilized itself, hundreds of times higher than those normally lethal. As for the activation of the immune system, there are available two classes of medicaments: immunizing vaccines and desensitizing vaccines, used against infections and against allergies, respectively.

According to the theory of the “clonal selection” (Burnet, 1959), the immune system arises from a heterogeneous population of embryonal cells, originally capable not only to recognize but also to attack most of the enormous variety of antigens existing within and without the organism containing the same. Before and immediately after the birth, these cells are subjected to a selection with an analogous meaning to the one which regulates, in the Darwinian conception of the phylogeny, the evolution of the living species (Darwin, 1859): those directed against the organism are eliminated, because they are dangerous for the survival, the others are preserved. The ability owned by living organisms to differentiate the oneself from the non-oneself comes, according to the above theory, from this ancestral process, mainly based on T and B cells. Each of them and of their progeny, the so-called clone, is able to individuate and attack a single antigen.

The theory of the “idiotypical immune network”, devised fifteen years after the one of the clonal selection (Jerne, 1974), ascribes a self-regulatory ability to the immune system due to a mechanism, a negative feed-back, wherein antibodies behave as the exogenous antigens, thus starting an antigenic reaction which slows down its production. This response has a latency of about 2-3 weeks, corresponding with the mobilization of the specific cell clone which is involved. During this latency, similar to the one of the primary response, the antibodies can manifest their effects. The antibody against the antigen acts, in turn, as an antigen, thus starting a series or interconnected reactions having a sinusoidal character.

It should be extremely useful being able to considerably improve the immune response of the organism towards causes/agents rising infections and/or allergies.

Namely, it should be extremely useful being able to provide vaccines which, for example, allow to activate the immune system in a lower time than that of vaccines commonly used in therapy and which, with the same dose administered, show to have an effectiveness at least equal, preferably better.

Vaccines having the above mentioned features could be, for example, advantageously administered at lower dosages than those normally employed for the prevention and/or the treatment of the same pathology, proving themselves, among other things, safer for the patient. Vaccines having the above innovative characteristics are not known.

There remains, therefore, the need to provide vaccines which allow to accelerate and ameliorate the immune and/or desensitizing response of the organism towards the causes/agents which rise infections and allergies, showing, at the same time, a high effectiveness and safety of use.

An object of the present invention is to give a proper answer to the need above mentioned.

These and other aims, which will better result from the following detailed description, have been attained by the Applicant, which has unexpectedly found that a proper quantity of a traditional vaccine/antigen, formulated in a sublingual pharmaceutical form (i.e. in a pharmaceutical formulation for a specific sublingual administration/intake) is able to give a proper answer to the problems above pointed out.

Therefore, a sublingual pharmaceutical composition including at least an antigen (for the prevention and/or the treatment of pathological conditions where an immunizing and/or desensitizing prevention and/or treatment are required, that is for the prevention and/or the treatment of pathologies due to infections and/or allergies), forms an object of the present invention, as reported in the appended independent claim.

Another object of the present invention is the use of said at least one antigen for the preparation of the above sublingual pharmaceutical composition, as reported in the appended independent claim.

Another object of the present invention is then a method for the preparation of the above composition, whose characteristics are reported in the appended independent claim.

Preferred embodiments of the present invention are reported in the appended dependent claims.

The present invention relates to a sublingual pharmaceutical composition including at least one antigen. Said at least one antigen is formulated so as to be absorbed by the oral mucosa, more specifically, by the sublingual mucosa.

Preferably, the antigen, or the mixture of antigens, is present in a freeze-dried form.

As for the dosage, the antigen, or the mixture of antigens, is usually present in a quantity corresponding with the one of, analogous compositions to be administered by injection. Preferably, said quantity is lower than the one of the corresponding compositions to be administered by injection.

Preferably, said quantity is between 20% and 100% by weight, with respect to the one of analogous compositions to be administered by injection; preferably, said quantity is between 30% and 95% by weight; more preferably, said quantity is between 40% and 85% by weight.

In a particularly preferred embodiment, said quantity is about 50% by weight with respect to the one of analogous compositions to be administered by injection.

In the sublingual pharmaceutical composition according to the present invention, the antigen, or the mixture of antigens, is formulated in admixture with at least a proper excipient typical of sublingual formulations. As non limiting example for the artisan skilled in the art, said excipients are selected from the group including: water-soluble inert excipients (such as, for example, mannitol, sorbitol, lactose); water-insoluble excipients which help the delivery of the active substance at the sublingual mucosa level (such as, for example, microcrystalline cellulose); sweeteners (such as, for example, aspartame, sodium saccharated); flavourings (such as, for example, peach, apricot, banana, strawberry, orange, mandarin flavours); lubricants (such as, for example, magnesium stearate, PEG 6000); taste correctors (such as, for example, sodium citrate); other excipients, additives, carriers commonly used in the formulation pharmaceutical art.

In a preferred embodiment of the invention, the sublingual composition includes the antigen, or the mixture of antigens, in admixture with at least one water-soluble excipient and/or at least one microcrystalline water-insoluble excipient with a disintegrating function.

Preferably, said water-soluble excipient is selected from mannitol and/or sorbitol and said microcrystalline water-insoluble excipient with a disintegrating function is microcrystalline cellulose.

In another preferred embodiment of the invention, said sublingual composition further includes at least one lubricant agent; preferably, said lubricant is magnesium stearate and/or PEG 6000 powder.

In a further preferred embodiment, said sublingual composition further includes at least a sweetening agent; preferably, said sweetener is aspartame and/or sodium saccharated.

The sublingual composition according to the present invention is utilizable for the administration of any type of antigen, both single and in a proper admixture with one or more other kinds of antigens. In this latter case, it is therefore possible to carry out a polyvalent vaccination with the administration of a single pharmaceutical form.

In a preferred embodiment of the invention, said at least one antigen has an immunizing activity towards pathologies due to infections.

In another preferred embodiment, said at least one antigen has a desensitizing activity towards pathologies due to allergies.

Preferred, absolutely not limiting examples of the applicative potential of the invention, are sublingual pharmaceutical compositions including at least an antigen, wherein said antigen has an immunizing activity towards a pathology selected from the group including: influenza, meningitis, rubella, tetanus, chickenpox.

### General Method for the Preparation of a Sublingual Pharmaceutical Composition of Vaccines.

The suspension/solution of antigens (such as, for example, the one contained in an injectable vial), added with inert excipients (for example mannitol, sorbitol, lactose) is freeze-dried. The powder obtained, finely divided, is mixed with a water-soluble excipient (for example mannitol, sorbitol) and a water-insoluble excipient (for example microcrystalline cellulose) which help the release at the sublingual mucosa level. The resulting mixture is further preferably added with a sweetener (for example aspartame, sodium saccharated) and/or a flavouring (for example peach, apricot, banana flavours) and/or a lubricant agent (for example magnesium stearate, PEG 6000) and/or a taste corrector (for example sodium citrate).

Absolutely non limiting examples of a sublingual composition are:

### Sublingual Composition of an Influenza Vaccine:

| | |
|---|---|
| Antigen freeze-dried product on mannitol | 25 mg |
| Mannitol | 30 mg |
| Microcrystalline cellulose | 20 mg |
| Aspartame | 5 mg |
| Peach flavour | 5 mg |
| Peg 6000 | 3 mg |
| Sodium citrate | 2 mg |

### Sublingual Composition of an Antimeningococcus Vaccine:

| | |
|---|---|
| Specific antigen freeze-dried product on | 30 mg |
| sorbitol | |
| Mannitol | 25 mg |
| Microcrystalline cellulose | 25 mg |
| Sodium saccharated | 5 mg |
| Banana flavour | 5 mg |
| Magnesium stearate | 3 mg |

Sodium citrate 2 mg

The sublingual compositions according to the present invention can be formulated in pharmaceutical forms suitable for the desired administration.

By mere way of example, said compositions are packaged, such as powder, in single dose sachets to be dissolved within the mouth, under the tongue, or are formulated in form of a sublingual tablet.

Particularly preferred is the form of sublingual tablet because of its convenience of use; for example, a fast release sublingual tablet.

The effectiveness of the compositions according to the present invention has been evaluated, in particular, on patients which refuse the injectable vaccines because of religious or ideological reasons, thinking that the forced introduction within the organism of substances unrelated to their natural composition is contrary to their principles.

The preference was given to those whose refusal could endanger adults involved in this event and the members of their family.

The experimentation was conducted according to what foreseen by law by decree Feb. 17, 1998, coordinated with the conversion law Apr. 8, 1998, no. 94, which, at subparagraph 1, states what follows: “the physician can, on his own direct responsibility and subject to information of the patient and acquisition of the approval of the same, employ a drug industrially produced for an indication, a route or a form of administration different from the authorized ones”. This prescription, establishes the above Decree, must be “in conformity with works appeared on scientific publications accredited in an international field”. Such works consist, in the particular case, of the wide documentation concerning the permeability of the sublingual mucosa to the drugs.

For this purpose, freeze-dried vaccines and formulated in fast disintegration tablets, containing half of the dose suggested for analogous vaccines injectable in vials, have been used.

Once a free and informed approval has been obtained, the following vaccines have been sublingually administered following the usual posology diagram, at doses reduced by half (50% by weight) with respect to those commonly used:

influenza vaccine;
antitetanic vaccine;
antirubella vaccine;
antimeningococcus vaccine;
antichickenpox vaccine.

In the volunteers subjected to experimentation, the hematic levels of the relative antibodies have been controlled, before the intake and after three weeks. The antibody response was excellent, in compliance with the one reported with the intramuscular injection of the whole dose.

The invention has also resulted applicable to desensitizing vaccines, which foresee the injection of progressively increasing doses of the antigen at progressively increasing doses (Abramson et al, 1995; Bousquet et al., 1998; Dykewitz et al, 1998; Jacobsen, 2001).

In this case, patients have been treated once a week for 5 months. Next, patients have been kept under treatment with the greatest dose once a week or a month, depending on the single requirements.

Also in these applications the antibody response was in compliance with the one reported with the intramuscular injection of the corresponding vaccines, also using lower doses of the vaccine itself.

From the results of the experimentations carried out, an innovative hypothesis unexpectedly takes shape and finds confirmation; i.e., that the immune reaction is started not only by the “extraneousness” of the antigen, but also by the “rate” with which the antigen penetrates into the organism or, in case of an endogenous origin thereof, by the rate with which it is produced or released by the compartments in which it is sequestered.

The sublingual composition of the present invention, allowing to modulate the absorption rate of the vaccine, has permitted to considerably improve the effectiveness of the same, both in terms of action rate and in terms of an increased safety for the patient.

1. Sublingual pharmaceutical composition including at least an antigen.

2. The composition according to claim 1, wherein said at least one antigen has an immunizing activity towards pathologies due to infections.

3. The composition according to claim 1, wherein said at least one antigen has a desensitizing activity towards pathologies due to allergies.

4. The composition according to claim 1, wherein said at least one antigen is in a freeze-dried form.

5. The composition according to claim 1, wherein said at least one antigen is present in a quantity between 20% and 100% by weight, with respect to the one of the corresponding compositions to be administered by injection.

6. The composition according to claim 5, wherein said quantity is between 30% and 95% by weight; preferably, is between 40% and 85% by weight; more preferably, is about 50% by weight.

7. The composition according to claim 1, further including at least an excipient selected from the group including: water-soluble inert excipients, water-insoluble excipients which help the release of the active substance at the sublingual mucosa level, sweeteners, flavourings, lubricants, taste correctors.

8. The composition according to claim 7, including at least a water-soluble inert excipient and/or at least a water-insoluble microcrystalline excipient as a disintegrant.

9. The composition according to claim 8, wherein said water-soluble inert excipient is selected from mannitol and/or sorbitol and said water-insoluble microcrystalline excipient is microcrystalline cellulose.

10. The composition according to claim 1, further including at least a lubricant agent: preferably, said lubricant is magnesium stearate and/or PEG 600 powder.

11. The composition according to claim 1, further including at least a sweetening agent: preferably, said sweetener is aspartame and/or sodium saccharinate.

12. Composition according to claim 1, wherein the antigen has an immune activity towards: influenza, tetanus, rubella, meningitis, chickenpox.

13. Use of a composition according to claim 1 for the preparation of a pharmaceutical formulation for the sublingual administration of at least an antigen.

14. The use according to claim 13, wherein said pharmaceutical formulation is a single dose sublingual sachet.

15. The use according to claim 13, wherein said pharmaceutical formulation is a sublingual tablet.

16. The use of at least one antigen for the preparation of a sublingual pharmaceutical composition for the immune treatment towards pathologies due to infections and/or for the desensitizing treatment towards pathologies due to allergies.

17. The use according to claim 16, wherein said pathologies are selected from the group including: influenza, tetanus, rubella, meningitis, chickenpox.
